(19) United States
(12) Patent Application Publication
(10) Pub. No.: US 2019/0376892 A1
(43) Pub. Date: Dec. 12, 2019

(54) FLUORESCENCE IMAGING DEVICE AND FLUORESCENCE IMAGING SYSTEM

(71) Applicant: Shimadzu Corporation, Kyoto (JP)

(72) Inventors: Akihiro ISHIKAWA, Kyoto (JP); Hiroyuki KITAMOTO, Kyoto (JP)

(21) Appl. No.: 16/477,293

(22) PCT Filed: Jan. 11, 2017
(86) PCT No.: PCT/JP2017/000662

§ 371 Date: Jul. 11, 2019

(51) Int. Cl. G01N 21/64; A61B 5/00

(52) U.S. Cl. CPC G01N 21/64; A61B 5/0077; A61B 5/0071

(57) ABSTRACT

This fluorescence imaging device 100 is provided with: an image acquisition unit 14 configured to acquire an image 12a of fluorescence generated by irradiating a fluorescent material administered to a subject P with excitation light; and an extraction unit 9 configured to extract, among images 12a of the fluorescence acquired by the image acquisition unit 14, an image 12a of the fluorescence in a predetermined time range including a predetermined timing for extracting the images 12a of the fluorescence image.

### TECHNICAL FIELD

The present invention relates to a fluorescence imaging device, and more particularly to a fluorescence imaging device and a fluorescence imaging system for acquiring an image of fluorescence generated by administering a fluorescent material to a subject and irradiating excitation light.

### BACKGROUND ART

Conventionally, a fluorescence imaging device is known in which an image of fluorescence generated by administering a fluorescent material to a subject and irradiating excitation light is acquired. Such a fluorescence imaging device is disclosed, for example, in Japanese Unexamined Patent Application Publication No. 2016-135253.

The fluorescence imaging device disclosed in the above-mentioned Japanese Unexamined Patent Application Publication No. 2016-135253 acquires a fluorescence image from the rising to the falling of the luminance of fluorescence by administering a fluorescent material to a subject and detecting fluorescence generated by irradiating excitation light. The fluorescence imaging device is configured to generate a fluorescence image by performing image processing of the acquired fluorescence image based on an index, such as, e.g., fluorescence intensity and a detection time of the fluorescence.

Such a fluorescence imaging device is used as a part of intraoperative support equipment to perform identification of the region of interest (affected part or the like) or confirmation of the state during surgery by making a display device or the like reproduce the image of the fluorescence recorded during the surgery. In addition, the above-described fluorescence imaging device is used to diagnose blood vessels, etc., of limbs by confirming recorded fluorescence images.

### PRIOR ART

### Patent Document

Patent Document 1: Japanese Unexamined Patent Application Publication No. 2016-135253

### SUMMARY OF THE INVENTION

### Problems to be Solved by the Invention

However, in the fluorescence imaging device disclosed in the above-mentioned Japanese Unexamined Patent Application Publication No. 2016-135253, after administration of a fluorescent material, a fluorescence image over the entire period covering the rising, the peak (maximum value of luminance), and the falling of the luminance of the fluorescence image is acquired. Therefore, there are problems that it takes time to search the image of the part that the user wants to confirm, such as the part where the fluorescence intensity of the region of interest is the strongest, it is difficult to confirm characteristic changes in luminance, etc., and the data amount of the image to be saved becomes large when saving to a recorder, etc. Note that in this specification, the “region of interest” refers to a region desired to be observed, such as, e.g., a tumor, of the fluorescence image.

The present invention has been made to solve the aforementioned problems, and one of objects of the present invention is to provide a fluorescence imaging device and a fluorescence imaging system capable of shortening the time required for searching a portion of a fluorescence image to be reproduced that a user wants to confirm, easily confirming characteristic changes in luminance, etc., and suppressing data amounts of images to be stored in a recorder or the like.

### Means for Solving the Problems

In order to achieve the above object, the fluorescence imaging device according to the first aspect of the present invention includes:
an image acquisition unit configured to acquire an image of fluorescence generated by irradiating a fluorescent material administered to a subject with excitation light; and
an extraction unit configured to extract, among images of fluorescence acquired by the image acquisition unit, an image of the fluorescence in a predetermined time range including a predetermined timing for extracting the image of the fluorescence.

In the fluorescence imaging device according to the first aspect of the present invention, as described above, the fluorescence imaging device is provided with an image acquisition unit configured to acquire an image of fluorescence and an extraction unit configured to extract the image of the fluorescence in a predetermined time range including a predetermined timing. With this, for example, it is possible to extract a fluorescence image in a predetermined time range including a predetermined timing required by the user, such as, e.g., a timing at which the fluorescence intensity becomes approximately the maximum value. Therefore, it is possible to extract an image of a portion where the user wants to confirm, such as a portion having the strongest fluorescence intensity of the region of interest. Also, by limiting the extraction range to a necessary and sufficient time range, it is possible to reduce the data amount of the fluorescence image. As a result, compared with the case in which a fluorescent material is administered to the subject and the image captured over the entire period from the rising to falling of the fluorescent generated by irradiating excitation light is reproduced and observed, it is possible to shorten the time required for searching a portion of a fluorescence image to be reproduced that a user wants to confirm, easily confirm characteristic changes in luminance, etc., and suppress data amounts of images to be stored in a recorder or the like.

In the fluorescence imaging device according to the first aspect of the present invention, preferably, the fluorescence imaging device further includes:
a timing detection means configured to detect the predetermined timing for extracting the image of the fluorescence,
wherein the extraction unit is configured to extract the image of the fluorescence in the predetermined time range including the timing for extracting the image of the fluorescence detected by the timing detection means. By configuring as described above, the extraction unit can determine a predetermined time range for extracting an image of fluorescence based on, for example, the timing for extracting the image of fluorescence detected by the timing detection means by a user operation or signal strength of the image of the fluorescence, so that it is possible to easily extract the fluorescence image in the predetermined time range.

In this case, preferably, the timing detection means is configured to detect the timing for extracting the image of the fluorescence based on signal strength of the fluorescence. By configuring as described above, it is possible to detect the timing at which the strength signal of the fluorescence has reached a predetermined value, the timing at which the change amount of the signal strength of the fluorescence turns from an increase to a decrease, etc., as the timing for extracting the image of the fluorescence. Therefore, it is possible to automatically determine the timing for extracting the highly visible image of the fluorescence.

More preferably, the timing detection means is configured to detect that it has become a timing at which the signal strength has reached a maximum value based on the signal strength of the fluorescence. By configuring as described above, it is possible to acquire the fluorescence image in the predetermined time range including the timing at which the signal strength of the fluorescence of the region of interest is the largest, so that the fluorescence high in visibility can be extracted assuredly.

In the configuration in which the timing for extracting an image of the fluorescence is detected based on the signal strength of the above-mentioned fluorescence, preferably, the timing detection means is configured to detect a timing at which the signal strength of the fluorescence becomes equal to or more than a threshold value based on the signal strength of the fluorescence. By configuring as described above, it is possible to extract the fluorescence image in the range in which the signal strength of the fluorescence is equal to or more than a predetermined value, so that it is possible to suppress the increase in the unnecessary data amount while preventing the extraction of the low visibility part of the fluorescence image.

In the configuration of extracting an image of the fluorescence in a predetermined time range including the timing for extracting an image of the fluorescence detected by the above timing detection means, preferably, the timing detection means is configured to detect the timing for extracting the image of the fluorescence based on an operation input by a user. By configuring as described above, the timing for acquiring the fluorescence image can be detected regardless of the signal strength of the fluorescence, so that it is possible to assuredly acquire the fluorescence image of the timing at which the user wants to acquire by reflecting the intention of the user.

In the fluorescence imaging device according to the first aspect of the present invention, preferably, the extraction unit is configured to extract, centering on the timing for extracting the image of the fluorescence, the image of the fluorescence in the predetermined time range from a first time before the timing for extracting the image of the fluorescence to a second time after the timing for extracting the image of the fluorescence. By configuring as described above, since the extraction can be performed including the progress before and after the timing to be extracted, the convenience for the user can be improved. Further, since different time ranges can be extracted before and after from the timing to be extracted, the range of the fluorescence image to be extracted can be changed according to the metabolism of the subject and/or the fluorescent material.

In the fluorescence imaging device according to the first aspect of the present invention, preferably, the image acquisition unit is provided with a first light source unit configured to emit excitation light and a first detection unit configured to detect the fluorescence. By configuring as described above, compared with the case in which a light source unit for emitting excitation light and a detection unit for detecting fluorescence are provided separately, the image of the fluorescence can be easily acquired.

In the fluorescence imaging device according to the first aspect of the present invention, preferably, the image acquisition unit is configured to acquire an image of visible light, and the fluorescence imaging device further comprises an image synthesizing unit configured to generate an image for reproduction in which the image of the fluorescence extracted by the extraction unit and the image of visible light reflected by the subject and extracted by the extraction unit are superimposed. By configuring as described above, it is possible to acquire an image in which the fluorescence image of the region of interest is synthesized on the image of the visible light. This makes it possible for the user to visually recognize an image that sees through the region of interest in the subject in the image of the visible light representing the appearance. As a result, the convenience of the user can be improved.

In this case, preferably, the image acquisition unit is further provided with a second light source unit for emitting the visible light and a second detection unit for detecting the visible light reflected by the subject. By configuring as described above, compared with the case in which a device for acquiring an image of fluorescence and a device for acquiring an image of visible light are separately provided, an image of fluorescence and an image of visible light of the same region of the subject can be easily acquired.

In the fluorescence imaging device according to the first aspect of the present invention, preferably, the fluorescence imaging device further includes: a temporary storage unit configured to temporarily store an image acquired by the image acquisition unit at a time corresponding to the predetermined time range. By configuring as described above, it is possible to store the image acquired not for the entire time period but for the time corresponding to the predetermined time range can be stored in the temporary storage unit for extraction by the extraction unit. Therefore, the storage capacity of the temporary storage unit can be minimized.

In the fluorescence imaging device according to the first aspect of the present invention, preferably, the fluorescence imaging device further includes: a recording unit configured to record an image extracted by the extraction unit and an image generated from the image extracted by the extraction unit. By configuring as described above, when recording an image extracted by the extraction unit and an image generated from the extracted image, there is no need to use an external recording device, so that the convenience of the fluorescence imaging device can be improved.

The fluorescence imaging system according to a second aspect of the present invention is provided with the fluorescence imaging device according to the first aspect of the present invention, a recording device configured to record an image for reproduction generated by the fluorescence imaging device, and a display device configured to display the image for reproduction.

As described above, the fluorescence imaging system according to the second aspect of the present invention is provided with the fluorescence imaging device according to the first aspect of the present invention, a recording device configured to record an image for reproduction, and a display device configured to display the image for reproduction. With this, for example, it is possible to extract an image for reproduction in a predetermined time range including a predetermined timing required by the user, such as the timing at which the fluorescence intensity becomes nearly the maximum value, etc. Therefore, it is possible to extract the image for reproduction of the portion that the user wants to confirm, such as the portion with the strongest fluorescence intensity of the region of interest. Also, by limiting the extraction range to a necessary and sufficient time range, it is possible to reduce the data amount of the image for reproduction. As a result, compared with the case in which a fluorescent material is administered and observation is performed by reproducing the image captured over the entire period from rising to falling of the fluorescence, it is possible to reduce the time for searching the portion where the user wants to check, and it is possible to easily confirm the change in characteristic luminance, etc., and it is possible to suppress the data amount of the image to be stored in a recorder or the like. In addition, since the display device is provided, it is possible to display an image for reproduction while recording.

### Effects of the Invention

According to the present invention, as described above, it is possible to provide a fluorescence imaging device and a fluorescence imaging system capable of reducing the time for searching a portion where a user wants to check among fluorescence images, easily confirming the change in characteristic luminance, etc., and suppressing the data amount of the image to be stored in a recorder or the like.

### BRIEF DESCRIPTION OF THE DRAWINGS

FIG. 1 is a schematic block diagram showing a fluorescence imaging system provided with a fluorescence imaging device according to a first embodiment of the present invention.

FIG. 2 is a diagram for explaining an image to be displayed on a display unit of the fluorescence imaging system provided with the fluorescence imaging device according to the first embodiment of the present invention.

FIG. 3 is a schematic diagram of an overall configuration of the fluorescence imaging system provided with the fluorescence imaging device according to the first embodiment of the present invention.

FIG. 4 is a diagram (cross-sectional view) showing a state in which a fluorescent material of the fluorescence imaging system provided with the fluorescence imaging device according to the first embodiment of the present invention generates fluorescence.

FIG. 5 is a diagram specifically showing an image to be displayed on a display unit of the fluorescence imaging system provided with the fluorescence imaging device according to the first embodiment of the present invention.

FIG. 6 is a graph for explaining a range of extracting the fluorescence image of the fluorescence imaging system provided with the fluorescence imaging device according to the first embodiment of the present invention.

FIG. 7 is a graph for explaining a method of determining the start point of the range of extracting the fluorescence image and the maximum value of the signal strength of the fluorescence of the fluorescence imaging system equipped with the fluorescence imaging device according to the first embodiment of the present invention.

FIG. 8 is a graph for explaining a method of determining an end point of a range for extracting the fluorescence image of the fluorescence imaging system provided with the fluorescence imaging device according to the first embodiment of the present invention.

FIG. 9 is a graph for explaining a method of determining a range for extracting the fluorescence image of the fluorescence imaging system provided with the fluorescence imaging device according to the second embodiment of the present invention.

FIG. 10 is a schematic block diagram showing a fluorescence imaging system provided with a fluorescence imaging device according to a third embodiment of the present invention.

FIG. 11 is a schematic block diagram showing a fluorescence imaging system provided with a fluorescence imaging device according to a fourth embodiment of the present invention.

FIG. 12 is a schematic block diagram showing a fluorescence imaging system provided with a fluorescence imaging device according to a first modification of the first embodiment of the present invention.

FIG. 13 is a schematic block diagram showing a fluorescence imaging system provided with a fluorescence imaging device according to a second modification of the first embodiment of the present invention.

FIG. 14 is a schematic block diagram showing a fluorescence imaging system provided with a fluorescence imaging device according to a third modification of the first embodiment of the present invention.

FIG. 15 is a diagram specifically showing an image displayed on a display unit of a fluorescence imaging system provided with a fluorescence imaging device according to a fourth modification of the first embodiment of the present invention.

### EMBODIMENTS FOR CARRYING OUT THE INVENTION

Hereinafter, embodiments embodying the present invention will be described with reference to the drawings.

### First Embodiment

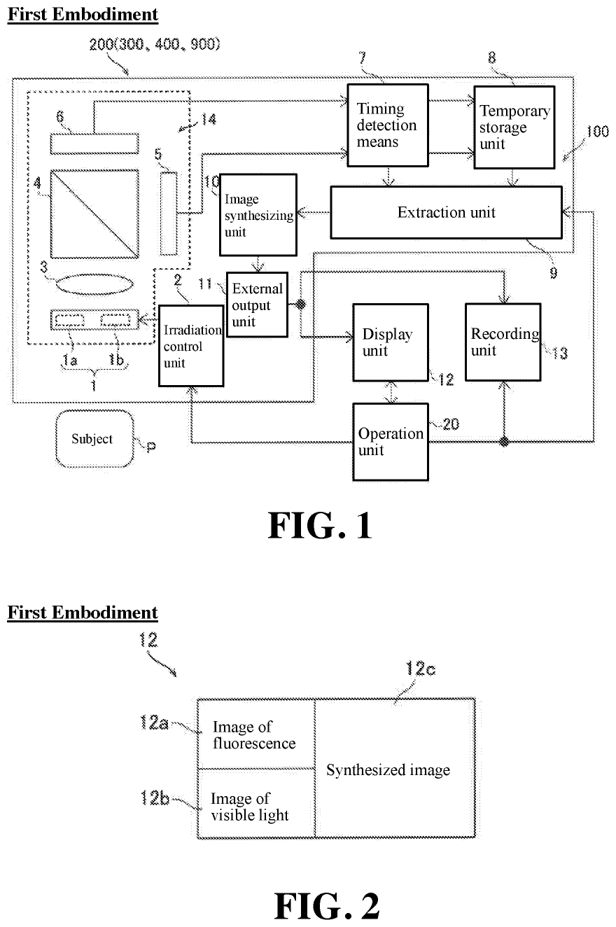

First, with reference to FIG. 1 to FIG. 7, a configuration of a fluorescence imaging system 200 provided with a fluorescence imaging device 100 according to a first embodiment will be described. Note that, in the first embodiment, the fluorescence imaging system 200 is a medical imaging device used for performing, for example, angiography or lymphangiography in surgery. Also note that the fluorescence imaging device 100 is used as a part of intraoperative support equipment (intraoperative support system) used during the surgery.

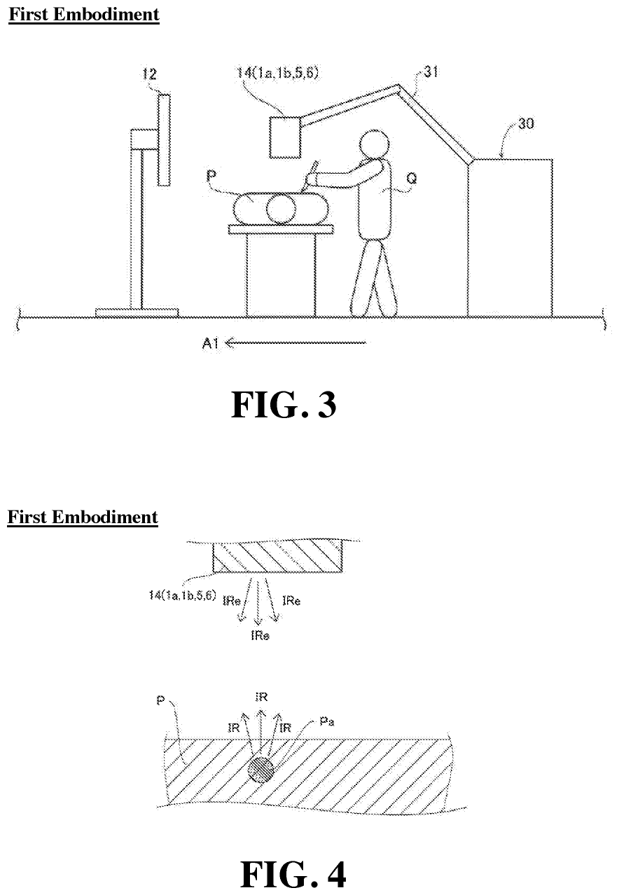

For example, the fluorescence imaging system 200 is used for confirming the position and shape of a blood vessel, a lymphatic vessel, and a lymph node of a subject P (patient) imaged in a surgery of a breast cancer sentinel lymph node by a user, such as, e.g., a surgeon Q (see FIG. 3).

### (Configuration of Fluorescence Imaging System)

As shown in FIG. 1, the fluorescence imaging system 200 according to the first embodiment is provided with a fluorescence imaging device 100, a display unit 12, a recording unit 13, and an operation unit 20. The fluorescence imaging device 100 is configured to extract an image of fluorescence and an image of visible light of the subject P and output an image obtained by synthesizing the extracted image of visible light and image of visible light. The detailed configuration of the fluorescence imaging device 100 will be described later. Note that the display unit 12 and the recording unit 13 are examples of the “display device” and the “recording device” recited in claims.

Further, as shown in FIG. 2, the display unit 12 displays an image 12a of fluorescence, an image 12b of visible light, and a synthesized image 12c output from the fluorescence imaging device 100.

Further, the recording unit 13 includes a storage device, such as, e.g., a storage element and an HDD, and is configured to record an image output from the fluorescence imaging device 100.

Further, the operation unit 20 is configured to accept an input operation to the fluorescence imaging device 100 by a user, such as, e.g., a surgeon Q and an operator of the fluorescence imaging system 200. The operation unit 20 is configured to operate the irradiation of light from the light source unit 1, the stop of irradiation, the adjustment of brightness and sensitivity, the display method of images to be displayed on the display unit 12, etc., based on the input operation.

### (Configuration of Fluorescence Imaging Device)

The fluorescence imaging device 100 according to the first embodiment is provided with an image acquisition unit 14 as shown in FIG. 1.

The image acquisition unit 14 is configured to acquire an image of a subject P. As the image acquisition unit 14, it is sufficient to have the light source unit 1 and detection units, such as, e.g., the visible light sensor 5 and the near infrared sensor 6 as a minimum configuration. In the first embodiment, the image acquisition unit 14 is provided with a light source unit 1, a zoom lens 3, a prism 4, a visible light sensor 5, and a near infrared sensor 6, and the image acquisition unit 14 is configured to capture images by itself. The zoom lens 3 and the prism 4 are arranged, as optical system members, between the light source unit 1, the visible light sensor 5, and the near infrared sensor 6. The zoom lens 3 is arranged between the light source unit 1 and the prism 4. The prism 4 is arranged between the zoom lens 3, the visible light sensor 5, and the near infrared sensor 6.

The light source unit 1 includes, for example, a light emitting diode (LED). The light source unit 1 is provided with a visible light source unit la for emitting visible light to a subject P (patient), an excitation light source unit 1b for emitting near infrared excitation light (hereinafter referred to as “excitation light IRe”) to a fluorescent agent Pa (see FIG. 4) in the body of the subject P. The visible light source unit la is an example of the “second light source unit” recited in claims. Note that the excitation light source unit 1b is an example of the “first light source unit” recited in claims. Further note that the fluorescent agent Pa is an example of the “fluorescent material administered to a subject” recited in claims. Further note that in the present specification, the “near infrared ray” means light having a wavelength longer than visible light, and, for example, is described as light having a wavelength within the range of 700 nm or more and 900 nm or less.

The fluorescent agent Pa is made of, for example, indocyanine green (ICG) which is a fluorescent dye. In the case of using indocyanine green (ICG) as a fluorescent agent Pa, the excitation light IRe is, for example, near infrared light having a wavelength of 800 nm or more and 820 nm or less. The excitation light IRe is irradiated to the indocyanine green, thereby generating near infrared fluorescence IR having a wavelength of about 830 nm from the indocyanine green. Further, the visible light irradiated from the visible light source unit la is reflected from the skin surface of the subject P as reflected light.

Further, the light source unit 1 is controlled by the irradiation control unit 2 of the fluorescence imaging device 100. The irradiation control unit 2 is configured as a control circuit, and is configured to control the irradiation of the light from the light source unit 1 (visible light, excitation light IRe), the stop of irradiation, etc., based on the input operation by the operation unit 20.

Further, to the zoom lens 3, the reflected light (visible light) from the skin surface of the subject P and the near infrared fluorescence IR generated from the fluorescent agent Pa are incident. Moreover, the zoom lens 3 adjusts the focal length of the visible light and the near infrared fluorescence IR.

The light from the zoom lens 3 is incident on the prism 4. The prism 4 has a function of separating the reflected light (visible light) from the skin surface of the subject P and the near infrared fluorescence IR.

Further, the image acquisition unit 14 is provided with a visible light sensor 5 for detecting the visible light separated by the prism 4. The visible light sensor 5 is configured by, for example, a charge coupled device (CCD) or a CMOS. Note that the visible light sensor 5 is an example of the “second detection unit” recited in claims.

Further, the image acquisition unit 14 is provided with a near infrared sensor 6 that detects the near infrared fluorescence IR generated by the excitation light IRe emitted from the excitation light source unit 1b. The near infrared sensor 6 is configured to be able to detect, for example, a near infrared ray having a wavelength within the range of 820 nm or more and 840 nm or less. The near infrared sensor 6 is configured by, for example, a CCD or a photomultiplier tube. Note that the near infrared sensor 6 is an example of the “first detection unit” recited in claims.

With such a configuration, the image acquisition unit 14 can simultaneously image the same imaging position of the subject P using the visible light and the excitation light IRe.

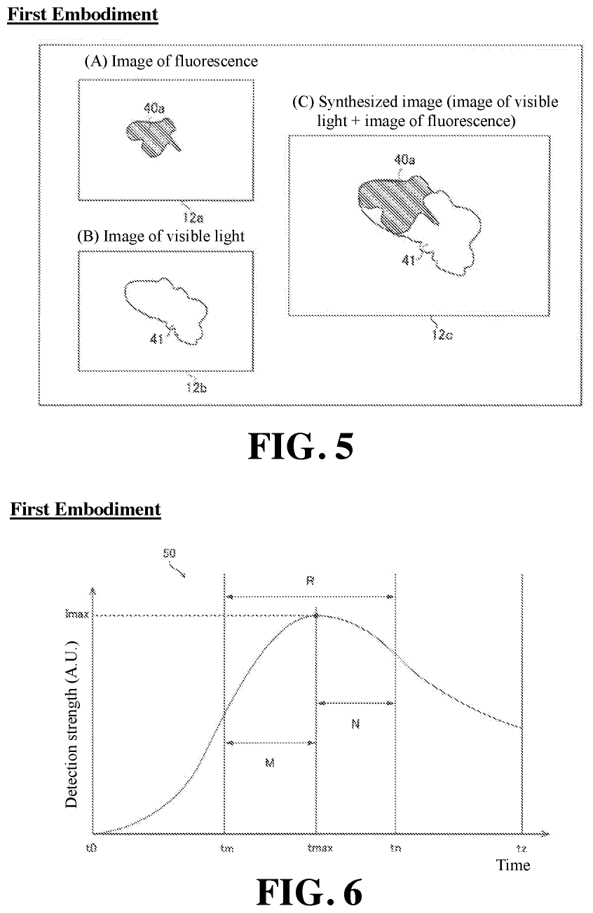

The fluorescence imaging device 100 is provided with a timing detection means 7. The timing detection means 7 detects the timing at which the extraction unit 9 extracts the image of the fluorescence of the subject P (see the figure (A) of FIG. 5) acquired by the near infrared sensor 6. The timing detection method will be described later.

Further, the fluorescence imaging device 100 is provided with a temporary storage unit 8. The temporary storage unit 8 includes a storage device, such as, e.g., a storage element and an HDD, and temporarily stores the image 12a of the fluorescence of the subject P acquired by the image acquisition unit 14 for the time corresponding to the predetermined time range R.

Further, the fluorescence imaging device 100 is provided with the extraction unit 9. The extraction unit 9 extracts the image 12a of the fluorescence in the predetermined time range R including the predetermined timing for extracting the image 12a of the fluorescence among the images 12a of the fluorescence generated by irradiating the fluorescent agent Pa administered to the subject P with the excitation light IRe.

The image data detected by the visible light sensor 5 and the near infrared sensor 6 is sent to the timing detection means 7 and the timing to be extracted is detected by the extraction unit 9. The timing detection means 7 sends the detected extraction timing to the extraction unit 9 and sends the image data to the temporary storage unit 8. The image data sent from the timing detection means 7 to the temporary storage unit 8 is temporarily stored by the temporary storage unit 8. The extraction unit 9 extracts the image data stored in the temporary storage unit 8 based on the timing for extracting the image sent by the timing detection means 7.

Note that the timing detection means 7, the temporary storage unit 8, the extraction unit 9, and the image synthesizing unit 10 may be individually configured by a CPU (Central Processing Unit), a memory, a GPU (Graphics Processing Unit), etc., and the timing detection means 7 and the extraction unit 9 may be configured as software in one CPU.

Further, the fluorescence imaging device 100 is provided with an image synthesizing unit 10. Here, in the first embodiment, as shown in FIG. 2, the image synthesizing unit 10 is configured to synthesize the image 12a of the fluorescence displayed using a color (for example, green) capable of being distinguished from the image 12b of visible light according to the signal strength of the near infrared fluorescence IR and the image 12b of the visible light that the visible light is imaged to form a synthesized image 12c. Specifically, the image synthesizing unit 10 is synthesized so as to generate a synthesized image 12c in which the image 12a of the fluorescence extracted by the extraction unit 9 and the image 12b of the visible light reflected by the subject P and extracted by the extraction unit 9 are superimposed. The image synthesizing unit 10 is configured, for example, as an image processing circuit. Note that the synthesized image 12c is an example of the “image for reproduction” recited in claims.

Further, the fluorescence imaging device 100 is provided with an external output unit 11. The external output unit 11 is configured to output the image 12a of the fluorescence extracted by the extraction unit 9, the image 12b of the visible light, and the synthesized image 12c synthesized by the image synthesizing unit 10 to a display unit 12 and a recording unit 13 which are provided outside the fluorescence imaging device 100.

Further, as shown in FIG. 3, the fluorescence imaging device 100 is provided with a device main body 30 in which the visible light source unit 1a, the excitation light source unit 1b, the visible light sensor 5, and the near infrared sensor 6 are provided. The visible light source unit 1a, the excitation light source unit 1b, the visible light sensor 5, and the near infrared sensor 6 are arranged in the image acquisition unit 14. Further, the device main body 30 is provided with an arm portion 31. The image acquisition unit 14 is attached to the tip end of the arm portion 31, and the image acquisition unit 14 is configured to be movable.

Further, the display unit 12 is provided separately from the device main body 30. For example, the display unit 12 is arranged in a direction facing the surgeon Q (user) (in the direction of the arrow A1), and is arranged at a height capable of visually recognizing the image displayed on the display unit 12 when the surgeon Q (user) performs the treatment of the subject P (patient).

As shown in FIG. 4, the fluorescent agent Pa inside the subject P generates near infrared fluorescence IR by the excitation light IRe irradiated by the excitation light source unit 1b provided in the image acquisition unit 14. The near infrared sensor 6 provided in the image acquisition unit 14 is configured to detect the near infrared fluorescence IR generated from the fluorescent agent Pa inside the subject P.

FIG. 5 is a conceptual diagram of an image to be displayed on the display unit 12. Note that a cancer cell vascularizes (forms) a large number of blood vessels to proliferate. Therefore, since there exist many blood vessels in the vicinity of the cancer cell, the vicinity of the cancer cell can be observed as a region labeled with the fluorescent agent Pa. The figure (A) of FIG. 5 shows the image 12a of fluorescence of the subject P, and the region of interest 40a is labeled by the fluorescent agent Pa. Further, the figure (B) of FIG. 5 shows the image 12b of visible light at the same site as the image 12a of the fluorescence of the subject P. Further, the figure (C) of FIG. 5 is a synthesized image 12c obtained by synthesizing the image 12a of the fluorescence and the image 12b of the visible light. In the fluorescence imaging device 100 according to the first embodiment, the arrangement of these images can be changed via the operation unit 20.

Next, with reference to FIG. 6 to FIG. 8, the image extraction processing of the fluorescence imaging device 100 will be specifically described.

FIG. 6 is a graph 50 showing the time change of the signal strength of the fluorescence of the region of interest 40a of the image 12a of the fluorescence. In the graph 50, the horizontal axis is a time and the vertical axis is the signal strength of the detected fluorescence.

In the fluorescence imaging device 100 according to the first embodiment, the extraction unit 9 is configured to extract the image 12a of the fluorescence in the predetermined time range R including the timing for extracting the image 12a of the fluorescence detected by the timing detection means 7. Specifically, the extraction unit 9 is configured to extract, centering on the timing for extracting the image 12a of the fluorescence, the image 12a of the fluorescence in the predetermined time range R from the predetermined time M before the timing for extracting the image 12a of the fluorescence to the predetermined time N after the timing for extracting the image 12a of the fluorescence. Further, the predetermined times M and N can be arbitrarily set by the user (surgeon Q, etc.). For example, the predetermined times M and N can be set in the range of 1 minute or less in total. Note that the predetermined times M and N are examples of the “first time” and the “second time” recited in claims.

Further, the timing detection means 7 is configured to detect the timing for extracting the image 12a of the fluorescence based on the signal strength of the fluorescence of the image 12a of the fluorescence. In the first embodiment, the timing detection means 7 is configured to detect that it has become the timing at which the signal strength has reached the maximum value based on the signal strength of the fluorescence.

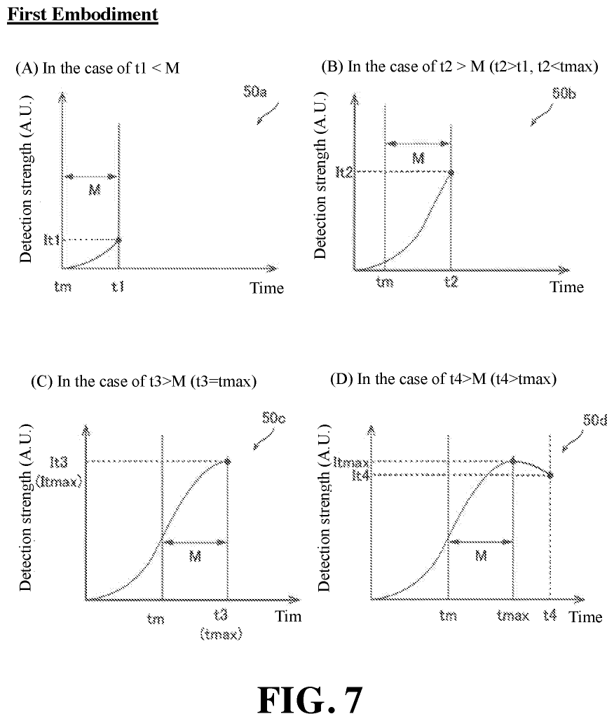

First, with reference to FIG. 6 and FIG. 7, the method of determining the maximum value of the signal strength of the fluorescence will be described. The timing detection means 7 is configured to detect the time “tmax” at which the signal strength of the region of interest 40a of the image 12a of the fluorescence becomes the maximum value “Itmax”. Specifically, the timing detection means 7 is configured to cut out an image one frame by one frame from the moving image captured by the near infrared sensor 6 and detect the maximum value and the maximum time by comparing the signal strengths of the fluorescence of the images for each frame. That is, every time one frame of the image 12a of the fluorescence is cut out, the maximum value of the signal strength of the fluorescence so far is compared with the signal strength of the fluorescence of the cutout image 12a of the fluorescence, and the larger one is taken as the maximum value. Also, the time at which the signal strength of the fluorescence becomes the maximum value is referred to as the time “tmax”. Here, the near infrared sensor 6 captures a moving image of high resolution high-definition image quality (for example, resolution: 1080 p (about 2.1 megapixel), 60 fps (frame per second)).

The figure (A) of FIG. 7 is a graph 50a showing the change in signal strength of fluorescence at the point of time when “t1” seconds have elapsed from the start of detection. At this point of time, since the signal strength “It1” of the fluorescence detected at “t1” seconds is the largest value, the maximum value of the signal strength of the fluorescence becomes “It1”. Further, the acquisition time of the image at which the signal strength of the light is the maximum value is “t1”.

The figure (B) of FIG. 7 is a graph 50b showing the change in signal strength of the fluorescence at the point of time when “t2” seconds have elapsed from the start of detection. At this point of time, since the signal strength “It2” of the fluorescence detected at “t2” seconds is the largest value, the maximum value of the signal strength of the fluorescence becomes “It2”. Further, the acquisition time of the image at which the signal strength of the light is the maximum value is “t2”.

The figure (C) of FIG. 7 is a graph 50c showing the change in signal strength of the fluorescence at the point of time when “t3” seconds have elapsed from the start of detection. At this point of time, since the signal strength “It3” of the fluorescence detected at “t3” seconds is the largest value, the maximum value of the signal strength of the fluorescence becomes “It3” (Itmax). Further, the acquisition time of the image at which the signal strength of the light is the maximum value is “t3” (tmax).

The figure (D) of FIG. 7 is a graph 50d showing the change in signal strength of the fluorescence at the point of time when “t4” seconds have elapsed from the start of detection. At this point of time, since the detection strength of “Itmax” detected at the time “tmax” seconds is larger than the signal strength “It4” of the fluorescence detected at “t4” seconds, the maximum value of the signal strength of the fluorescence becomes “Itmax”. Further, the acquisition time of the image at which the signal strength of the light is the maximum value is the time “tmax(t3)”. Thereafter, when the signal strength of the fluorescence changes as shown in FIG. 6, “t3” can be determined as the time “tmax”. Then, the timing detection means 7 outputs the time “tmax” to the extraction unit 9.

Next, with reference to FIG. 6 to FIG. 8, the method of determining the predetermined time range R that the extraction unit 9 extracts will be described. The extraction unit 9 is configured to acquire the image 12a of the fluorescence in the predetermined time range R which is determined by “tm” which is predetermined M seconds before “tmax” and “tn” which is predetermined N seconds after “tmax”, centering on the timing (tmax) detected by the timing detection means 7.

First, with reference to the figure (A) of FIG. 7 to the figure (D) of FIG. 7, the method of determining “tm” which is a predetermined time which is M seconds before the time “tmax” will be described. The graph 50a shown in the figure (A) of FIG. 7 is a graph showing a case where the time t1 from the start of detection is smaller than the predetermined time M. Since “t1” is less than or equal to the predetermined time M, “tm” is the detection start time.

Further, the figure (B) of FIG. 7 is a graph 50b showing the state in which the detection has progressed to the detection time “t2”. Specifically, it shows the state in which the time “t2” is larger than the predetermined times “M” and “t1” and smaller than the time “tmax”. Therefore, the time “tm” is a time that is the predetermined M seconds before “t2”.

Further, the figure (C) of FIG. 7 is a graph 50c showing the state in which the detection has progressed until the time “t3” when the signal strength of the fluorescence becomes the maximum value. Since it is the time when the signal strength of the fluorescence becomes the maximum value, the time “t3” and the time “tmax” become equal. Therefore, the time “tm” is a time that is the predetermined M seconds before the time “t3”.

Further, the figured (D) of FIG. 7 is a graph 50d showing the state in which the signal strength of the fluorescence has passed the maximum value and the detection has progressed to the time “t4” when it has started to decrease. Since the time “tm” is a time which is predetermined time M seconds before the time (tmax) at which the signal strength of the fluorescence becomes the maximum value, “tm” is a time which is M seconds before the time “tmax”.

Therefore, the time “tm” is changed according to the detection time, and is fixed to the time before the predetermined time M seconds from the time “tmax” after the time (tmax) at which the signal strength of the fluorescence is the maximum value is determined.

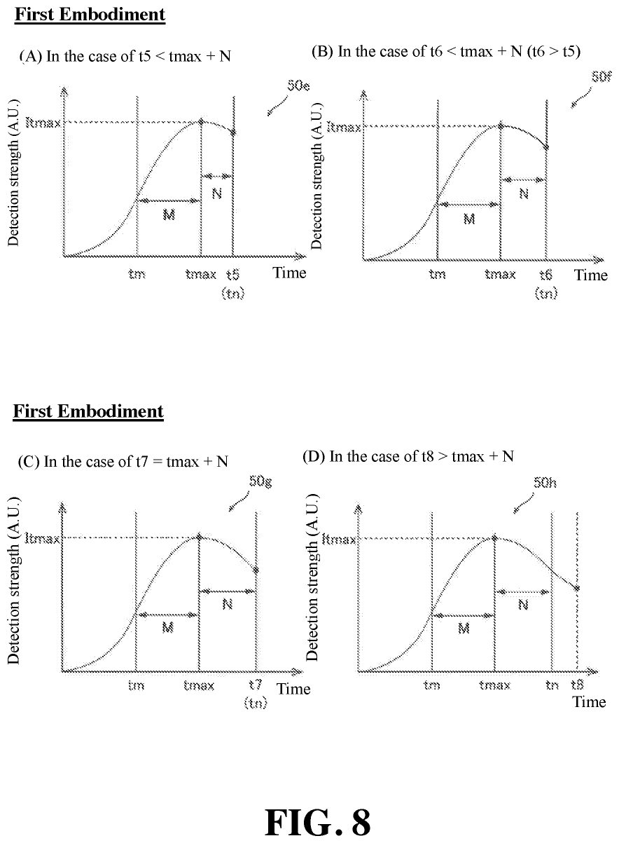

Next, with reference to the figures (A) to (D) of FIG. 8, the method of determining the time “tn” which is the time after the determined time N seconds from the time (tmax) at which the signal strength of fluorescence becomes the maximum value will be described. The graph 50e shown in the figure (A) of FIG. 8 is a graph showing the case in which the elapsed time “t5” from the start of detection is smaller than the time elapsed from the predetermined time N seconds from the time “tmax”. Since the time “t5” is equal to or less than N seconds from the time “tmax”, the time “tn” is the same as the time “t5”.

Further, the figure (B) of FIG. 8 is a graph 50f showing the state in which the detection has progressed to the detection time “t6”. Specifically, the time “t6” is larger than the time “t5” and smaller than the time elapsed by the predetermined time N seconds from the time “tmax”. Therefore, the time “tn” is the same time as the time “t6”.

Further, the figure (C) of FIG. 8 is a graph 50g showing the state in which the detection time “t7” has progressed to the same time as the time when it has advanced from the “tmax” by the predetermined time N. Since the time “t7” and the time advanced by N seconds from the time “tmax” are equal, the time “t7” and the time “tn” become equal.

Further, the figure (D) of FIG. 8 is a graph 50h showing the state in which the detection has progressed to the time at which the detection time “t8” has progressed from the time “tmax” by the predetermined time N seconds or more. Since the time “t8” has progressed from “tmax” by the predetermined time N seconds or more, the time “tn” is a time after the time “tmax” by the N seconds.

Therefore, the time “tn” is changed with time until the predetermined time N seconds have elapsed from the time (tmax) at which the signal strength of the fluorescence becomes the maximum value, and is fixed to the time after the predetermined time N seconds from the time “tmax” after the predetermined time N seconds have elapsed from the time “tmax”.

By the above processing, the extraction unit 9 extracts the image 12a of the fluorescence in the predetermined time range R (M+N) from the temporary storage unit 8 based on the predetermined timing (tmax) acquired from the timing detection means 7, and outputs the image 12a to the image synthesizing unit 10. It is enough that the temporary storage unit 8 can temporarily store the image 12a of the fluorescence for the maximum value of the predetermined time range R(M+N). That is, the temporary storage unit 8 erases the data older than M seconds from the time “tmax” and temporarily stores the newly acquired image.

### Effects of First Embodiment

In the first embodiment, the following effects can be obtained.

In the first embodiment, as described above, the fluorescence imaging device 100 is provided with: the image acquisition unit 14 for acquiring the image 12a of the fluorescence generated by irradiating excitation light IRe to the fluorescent agent Pa administered to the subject P; and the extraction unit 9 for extracting the image 12a of the fluorescence in the predetermined time range R including the predetermined timing for extracting the image 12a of the fluorescence among the image 12a of the fluorescence acquired by the acquisition unit 14. With this, it is possible to extract the synthesized image 12c in the predetermined time range R including the timing (tmax) at which the fluorescence intensity is the maximum value, so that it is possible to extract the synthesized image 12c of the portion where the fluorescence intensity of the region of interest 40a is the strongest. Further, since the range to be extracted is limited to the determined time range R, the data amount of the synthesized image 12c can be reduced. As a result, compared with the case in which a fluorescent material Pa is administered and observation is performed by reproducing the image captured over the entire period from rising to falling of the fluorescence, it is possible to shorten the search time for reproducing the synthesized image 12c in the predetermined time range R including the timing (tmax) at which the signal strength of the fluorescence of the region of interest 40a that the surgeon Q wants to confirm is the maximum value, easily confirm characteristic luminance changes, etc., and suppress the data amount of the synthesized image 12c stored in the recording unit 13.

Further, in the first embodiment, as described above, the fluorescence imaging device 100 is further provided with a timing detection means 7 for detecting the predetermined timing for extracting the image 12a of the fluorescence, and the extraction unit 9 is configured to extract the image 12a of the fluorescence in the predetermined time range R including the timing for extracting the image 12a of the fluorescence detected by the timing detection means 7. With this, since the determined time range R in the image 12a of the fluorescence to be extracted can determine the timing for extracting the image 12a of the fluorescence by the operation of the surgeon Q and/or the intensity of the signal of the image 12a of the fluorescence, the image 12a of the fluorescence of the predetermined time range R can be easily extracted.

Further, in the first embodiment, as described above, the timing detection means 7 is configured to detect the timing for extracting the image 12a of the fluorescence based on the signal strength of fluorescence. With this, since it is possible to detect the timing at which the intensity signal of the fluorescence has reached the predetermined value, the timing at which the amount of change in signal strength of the fluorescence has turned from an increase to a decrease, etc., as the timing for extracting the image 12a of the fluorescence. Therefore, the timing for extracting the highly visible image 12a of the fluorescence can be determined automatically.

Further, in the first embodiment, as described above, the timing detection means 7 is configured to detect that it has become the timing at which the signal strength has reached the maximum value based on the signal strength of the fluorescence. This makes it possible to acquire the image 12a of the fluorescence in the predetermined time range R including the timing (tmax) at which the signal strength of the region of interest 40a is the maximum, so that the highly visible image 12a of the fluorescence can be extracted with assuredly.

Further, in the first embodiment, as described above, the extraction unit 9 is configured to extract, centering on the timing (tmax) for extracting the image 12a of the fluorescence, the image 12a of the fluorescence in the predetermined time range R from the predetermined time M before the timing for extracting the image 12a of the fluorescence to the predetermined time N after the timing for extracting the image 12a of the fluorescence. With this, since the extraction can be performed including the progress before and after the timing for extraction, the convenience for the surgeon Q can be improved. Further, since different time ranges can be extracted before and after the extraction timing, the range of the image 12a of the fluorescence to be extracted can be changed according to the metabolism of the subject P or the fluorescent agent Pa.

Further, in the first embodiment, as described above, the image acquisition unit 14 is provided with an excitation light source unit 1b for emitting excitation light IRe and a near infrared sensor 6 for detecting near infrared fluorescence IR. With this, compared with the case of separately providing the excitation light source unit 1b for emitting excitation light IRe and the near infrared sensor 6 for detecting the near infrared fluorescence IR, the image 12a of the fluorescence can be easily acquired.

Further, in the first embodiment, as described above, the image acquisition unit 14 is configured to acquire the image 12b of visible light, and the fluorescence imaging device is further provided with the image synthesizing unit 10 for generating the synthesized image 12c in which the image 12a of the fluorescence extracted by the extraction unit 9 and the image 12b of the visible light extracted by the extraction unit 9 are superimposed. With this, it is possible to acquire the synthesized image 12c in which the image 12b of the visible light and the image 12a of the region of interest 40a are synthesized, so that an image that looks through the region of interest 40a in the subject P in the image 12b of the visible light that shows the appearance can be visually recognized by the surgeon Q. As a result, the convenience of the surgeon Q can be improved.

Further, in the first embodiment, as described above, the image acquisition unit 14 is further provided with the visible light source unit la for emitting visible light and a visible light sensor 5 for detecting the visible light reflected by the subject P. With this, compared with the case in which the device for acquiring the image 12a of fluorescence and the device for acquiring the image 12b of the visible light are separately provided, the image 12a of the fluorescence and the image 12b of the visible light of the same site of the subject P can be easily acquired.

Further, in the first embodiment, as described above, the temporary storage unit 8 for temporarily storing the image acquired by the image acquisition unit 14 at a time corresponding to the predetermined time range R is further provided. With this, since the image acquired for the time corresponding to the determined time range R can be stored in the temporary storage unit 8, the storage capacity of the temporary storage unit 8 can be minimized.

Further, in the first embodiment, as described above, the fluorescence imaging system 200 is provided with the fluorescence imaging device 100, the display unit 12 for displaying the synthesized image 12c generated by the fluorescence imaging device 100, the recording unit 13 for recording the synthesized image 12c, and the operation unit 20. With this, it is possible to extract the synthesized image 12c in the predetermined time range R including the timing (tmax) at which the fluorescence intensity is the maximum value, so that it is possible to extract the synthesized image 12c of the portion where the fluorescence intensity of the region of interest 40a is the strongest. Further, since the range to be extracted is limited to the determined time range R, the data amount of the synthesized image 12c can be reduced. As a result, compared with the case in which a fluorescent material Pa is administered and observation is performed by reproducing the image captured over the entire period (the period from the time “t0” to the time “tz” in FIG. 6) from rising to falling of the fluorescence, it is possible to shorten the time for reproducing the synthesized image 12c in the predetermined time range R including the timing (tmax) at which the signal strength of the fluorescence of the region of interest 40a is the maximum value, easily confirm characteristic luminance changes, etc., and suppress the data amount of the synthesized image 12c stored in the recording unit 13. In addition, since the display unit 12 is provided, the synthesized image 12c can be displayed while being recorded.

### Second Embodiment

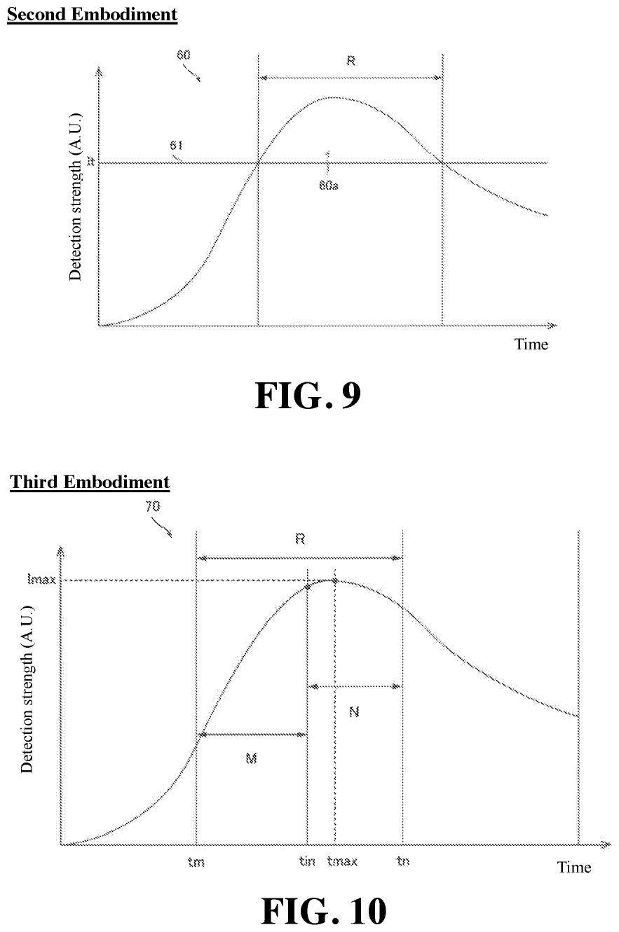

Next, with reference to FIG. 1 and FIG. 9, the configuration of the fluorescence imaging system 300 according to the second embodiment will be described. In the fluorescence imaging system 300 according to the second embodiment, unlike the first embodiment in which the timing detection means 7 is configured to detect that it has become the timing at which the signal strength of the fluorescence has reached the maximum value based on the signal strength of the fluorescence, the timing detection means 7 is configured to detect the timing at which the signal strength of the fluorescence becomes equal to or greater than a threshold value based on the signal strength of the fluorescence. Note that the same configurations as those in the first embodiment are illustrated by allotting the same reference symbols as in the first embodiment, and the description thereof will be omitted.

FIG. 9 is a graph 60 showing a predetermined time range R detected by the timing detection means 7 in the fluorescence imaging system 300 according to the second embodiment. The straight line 61 indicates a threshold value “It” previously set by the surgeon Q or the like. In the second embodiment, the timing detection means 7 is configured to detect the timing at which the signal strength of the fluorescence becomes equal to or greater than the threshold value “It” based on the signal strength of the fluorescence. Specifically, the timing detection means 7 sets the determined time range R so as to be the portion of the region 60a beyond the threshold value “It” formed by the graph 60 and the straight line 61. This allows the extraction unit 9 to extract the image in the predetermined time range R exceeding the threshold value “It”, such as the portion of the region 60a.

The other configurations of the fluorescence imaging system 300 according to the second embodiment are the same as those of the fluorescence imaging system 200 in the first embodiment.

### (Effects of Second Embodiment)

In the second embodiment, the following effects can be obtained.

In the second embodiment, as described above, the timing detection means 7 is configured to detect the timing at which the signal strength of the fluorescence becomes equal to or greater than the threshold value “It” based on the signal strength of the fluorescence. As a result, since the image 12a of the fluorescence of the region 60a in which the signal strength of the fluorescence is equal to or more than “It” can be extracted, it is possible to suppress the increase in the unnecessary data amount by avoiding the low visibility portion of the image 12a of the fluorescence.

Further, the other effects of the fluorescence imaging system 300 according to the second embodiment are similar to those of the fluorescence imaging system 200 according to the first embodiment.

### Third Embodiment

Next, with reference to FIG. 1 and FIG. 10, the configuration of the fluorescence imaging system 400 according to the third embodiment will be described. The fluorescence imaging system 400 according to the third embodiment is configured, unlike the first embodiment in which the timing for extracting the image 12a of the fluorescence is detected based on the signal strength of the fluorescence, to detect the timing for extracting the image 12a of the fluorescence based on the operation input of the surgeon Q. Note that the same configurations as the first embodiment are illustrated by allotting the same reference symbols as the first embodiment, and the description will be omitted.

FIG. 10 is a graph 70 showing the predetermined time range R detected by the input of a user in the fluorescence imaging system 400 according to the third embodiment. The timing detection means 7 is configured to detect the timing for extracting the image 12a of the fluorescence based on the operation input of the user. In the fluorescence imaging system 400 according to the third embodiment, the predetermined timing for detecting does not have to be strictly the timing (tmax) of the peak (Itmax), the scene to be reproduced is acquired as the input timing (tin) and the predetermined time range R determined by M and N before and after the input timing (tin) is acquired.

Further, the other configurations of the fluorescence imaging system 400 according to the third embodiment are the same as those of the fluorescence imaging system 200 according to the first embodiment.

### (Effects of Third Embodiment)

In the third embodiment, the following effects can be obtained.

In the third embodiment, as described above, the timing detection means 7 is configured to detect the timing for extracting the image 12a of the fluorescence based on the operation input of the surgeon Q. With this, the timing for acquiring the image 12a of the fluorescence can be detected regardless of the signal strength of the fluorescence, it is possible to assuredly acquire the image 12a of the fluorescence of the timing that the surgeon Q wants to acquire, reflecting the intention of the surgeon Q.

Further, the other effects of the fluorescence imaging system 400 according to the third embodiment are the same as those of the fluorescence imaging system 200 according to the first embodiment.

### Fourth Embodiment

Next, with reference to FIG. 11, the configuration of the fluorescence imaging system 500 according to the fourth embodiment will be described. In the fluorescence imaging system 500 according to the fourth embodiment, unlike the first embodiment configured to record the image 12a of the fluorescence, the image 12b of the visible light, and the synthesized image 12c by the recording unit 13 provided outside the fluorescence imaging device 100, the fluorescence imaging device 100 is further provided with the recording unit 13.

As shown in FIG. 11, in the fluorescence imaging system 500 according to the fourth embodiment, the fluorescence imaging device 100 is further provided with a recording unit 13. The image synthesizing unit 10 is configured to send the image 12a of the fluorescence, the image 12b of the visible light, and the synthesized image 12c to the external output unit 11, and send the image 12a of the fluorescence, the image 12b of the visible light, and the synthesized image 12c to the recording unit 13.

The other configurations of the fluorescence imaging system 500 according to the fourth embodiment are the same as those of the fluorescence imaging system 200 according to the first embodiment.

### (Effects of Fourth Embodiment)

In the fourth embodiment, the following effects can be obtained.

In the fourth embodiment, as described above, the fluorescence imaging device 100 is further provided with the recording unit 13. With this, when recording the image (image 12a of the fluorescence, image 12b of the visible light) extracted by the extraction unit 9 and the image (synthesized image 12c) generated from the extracted image, since it is not necessary to use an external recording device, the convenience of the fluorescence imaging device can be improved.

Further, the other effects of the fluorescence imaging system 500 according to the fourth embodiment are the same as those of the fluorescence imaging system 200 according to the first embodiment.

### Modified Embodiment

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the aforementioned first to fourth embodiments, an example is shown in which the fluorescence imaging system is configured as an intraoperative support system used for angiography and lymphangiography during surgery, but the present invention is not limited thereto. For example, the fluorescence imaging system may be placed in a doctor's office and used for a diagnosis that does not require surgery, such as a diagnosis of skin cancer.

Further, in the first to fourth embodiments, an example is shown in which a light emitting diode is included in the excitation light source unit 1b for irradiating near infrared fluorescence IR, but the present invention is not limited to this. That is, the excitation light source unit 1b may include a light emitting member other than a light emitting diode. For example, a bulb light source, such as, e.g., a halogen, may be provided in the excitation light source unit 1b, or any light source may be used as long as the light source emits the excitation light.

Further, in the first to fourth embodiments, an example is shown in which the fluorescent agent Pa is indocyanine green, but the present invention is not limited to this. That is, the fluorescent agent Pa may be a fluorescent agent other than indocyanine green.

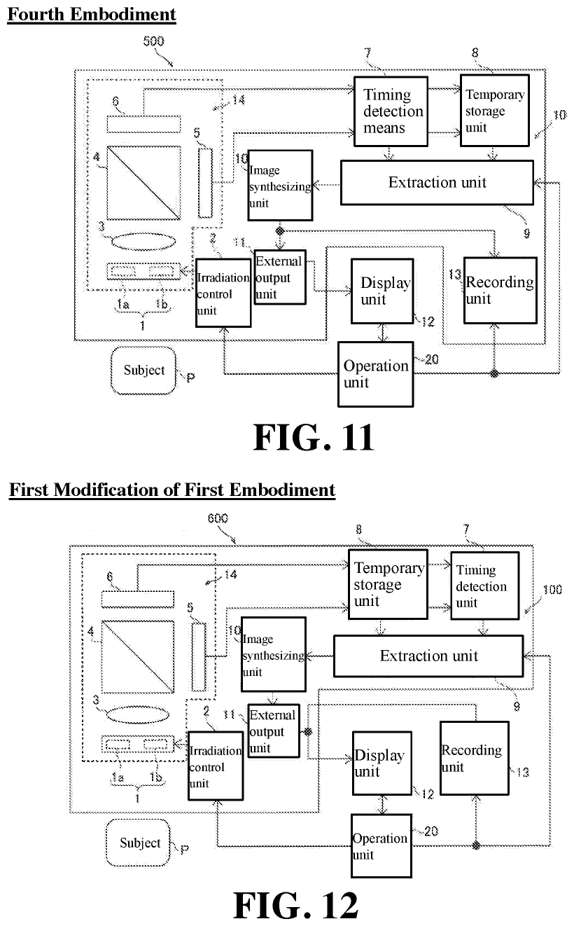

Further, in the first to fourth embodiments, an example is shown in which an image captured at the timing by the timing detection means 7 is temporarily stored, but the present invention is not this. For example, like the fluorescence imaging system 600 according to the first modification of the first embodiment shown in FIG. 12, it may be configured to detect the image stored in the temporary storage unit 8 at the timing for extracting by the timing detection means 7. However, when configured as described above, at the time of performing the timing detection of the image to be extracted by the timing detection means 7, it is necessary to read the image data from the temporary storage unit 8, so that the processing of the timing detection takes time as compared with the configuration of FIG. 1. Therefore, it is preferable to use the configuration of FIG. 1.

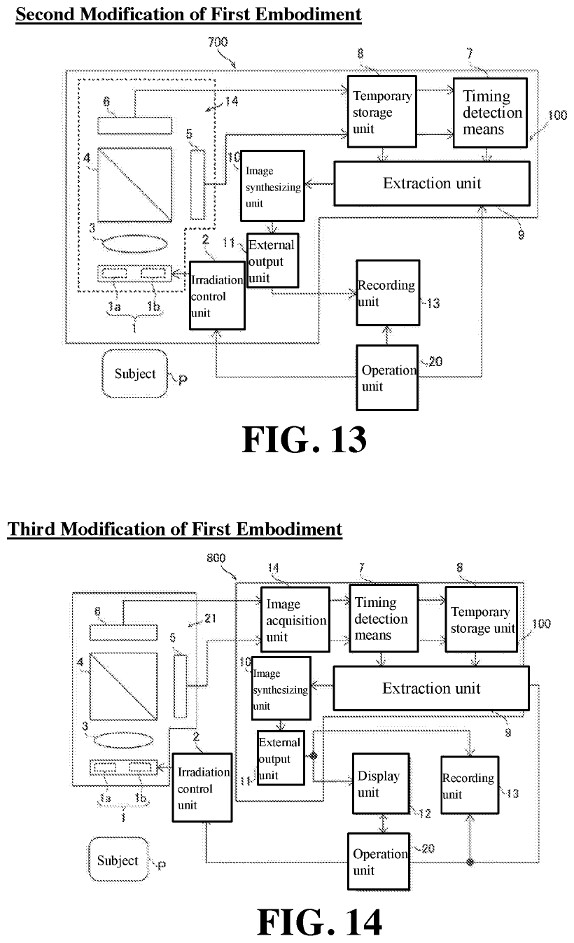

Further, in the first to fourth embodiments, an example is shown in which the image 12a of the fluorescence, the image 12b of the visible light, and the synthesized image 12c are displayed by the display unit 12 and recorded by the recording unit 13, but the present invention is not limited thereto. For example, like the fluorescence imaging system 700 according to the second embodiment of the first embodiment shown in FIG. 13, it may be configured to perform only the recording by the recording unit 13.

Further, in the first to fourth embodiments, an example is shown in which the excitation light IRe of near infrared ray is irradiated, but the present invention is not limited to this. The excitation light IRe to be irradiated to the subject P may be light of an excitable wavelength according to the fluorescent agent Pa administered to the subject P.

Further, in the first to fourth embodiments, an example is shown in which the image acquisition unit 14 captures the image 12b of the fluorescence and the image 12b of the visible light, but the present invention is not limited thereto. For example, like the fluorescence imaging system 800 according to the third modification of the first embodiment shown in FIG. 14, the image acquisition unit 14 may be configured to acquire the image 12a of the fluorescence and the image 12b of the visible light captured by the imaging device 21 provided externally. In this case, the image acquisition unit 14 is configured as a data input unit for receiving an input of image data, and is connected to the imaging device 21 so as to be able to receive data by wire or wirelessly.

Further, in the first to fourth embodiments, an example is shown in which the timing detection means 7 detects the timing for extracting the image 12a of the fluorescence based on the signal strength of the fluorescence of the region of interest 40a of the image 12a of the fluorescence, but the present invention is not limited to this. For example, it may be configured such that the timing for extracting the image 12a of the fluorescence is determined based on the signal strength of the fluorescence of the entirety of the image 12a of the fluorescence (entire pixel region).

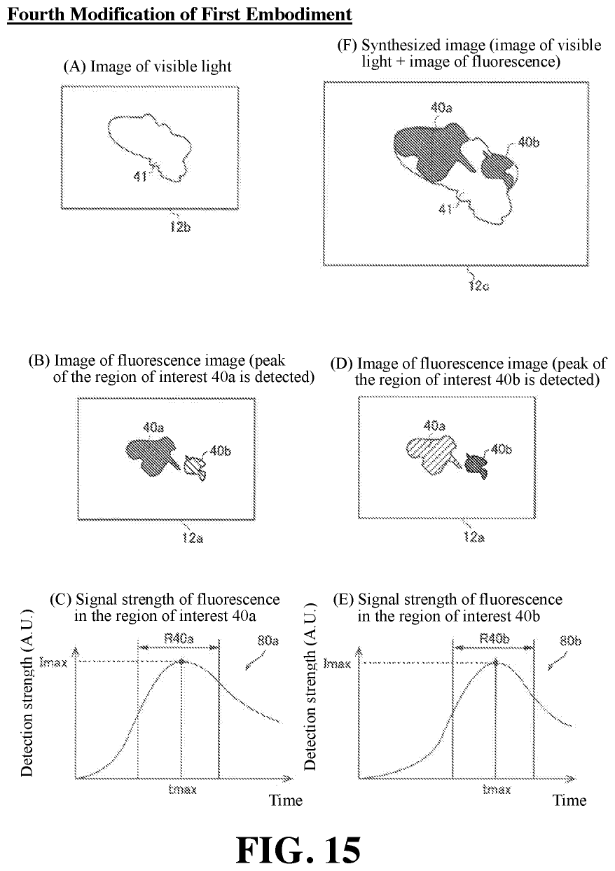

Further, in the first to fourth embodiments, an example is shown in which the timing detection means 7 detects the timing for extracting the image 12a of the fluorescence based on the signal strength of the fluorescence of the region of interest 40a of the image 12a of the fluorescence, but the present invention is not limited to this. For example, as shown in FIG. 15, the timing detection means 7 of the fluorescence imaging system 900 according to the fourth modification of the first embodiment shown in FIG. 1 may decide the timing for extracting the image 12a of the fluorescence based on the signal strength (the figures (C) and (E) of FIG. 15) of the fluorescence of the region of interest 40a and the region of interest 40b in the image 12a of the fluorescence. At that time, for example, it may be configured such that the extraction unit 9 extracts the image in the predetermined time range R (R40a and R40b) in which the signal strength of the fluorescence of each of the region of interest 40a and the region of interest 40b is near the maximum value, and the image synthesizing unit 10 synthesizes the image 12b of visible light (the figure (A) of FIG. 15), the image 12a of the fluorescence (figures (C) and (E) of FIG. 15) of the region of interest 40a and the region of interest 40b respectively into one image to generate a synthesized image 12c (figure (F) of FIG. 15). Note that the plurality of regions of interest is usually set at two to three places, for example, in surgery and the like. Further, the region of interest can be set up to eight places.

In the first to fourth embodiments, an example is shown in which the image 12a of the fluorescence, the image 12b of the visible light, and the synthesized image 12c are displayed on the display unit 12, but the present invention is not limited to this. For example, only the synthesized image 12c may be displayed. Also, for example, the image 12a of the fluorescence and the image 12b of the visible light may be displayed without synthesizing the images. In that case, the fluorescence imaging device may not have the image synthesizing unit 10. Also, for example, only the image 12a of the fluorescence may be displayed. In that case, the fluorescence imaging device is not required to provide the visible light source unit la, the visible light sensor 5, and the image synthesizing unit 10.

### DESCRIPTION OF REFERENCE SYMBOLS

1a: visible light source unit (second light source unit)
1b: excitation light source unit (first light source unit)
5: visible light sensor (second detection unit)
6: near infrared sensor (first detection unit)
7: timing detection means
8: temporary storage unit
9: extraction unit
10: image synthesizing unit
12: display unit
12a: image of the fluorescence
12b: image of the visible light
12c: image for reproduction
13: recording unit
14: image acquisition unit
100: fluorescence imaging device
200, 300, 400, 500, 600, 700, 800, 900: fluorescence imaging system
It: threshold value
M: predetermined time (first time)
N: predetermined time (second time)
P: subject
Pa: fluorescent agent (fluorescent material administered to the subject)
Q: surgeon (user)
R: predetermined time range

1. A fluorescence imaging device comprising:
an image acquisition unit configured to acquire an image of fluorescence generated by irradiating a fluorescent material administered to a subject with excitation light; and
an extraction unit configured to extract, among images of fluorescence acquired by the image acquisition unit, an image of the fluorescence in a predetermined time range including a predetermined timing for extracting the image of the fluorescence.

2. The fluorescence imaging device as recited in claim 1, further comprising:
a timing detection means configured to detect the predetermined timing for extracting the image of the fluorescence,
wherein the extraction unit is configured to extract the image of the fluorescence in the predetermined time range including the timing for extracting the image of the fluorescence detected by the timing detection means.

3. The fluorescence imaging device as recited in claim 2,
wherein the timing detection means is configured to detect the timing for extracting the image of the fluorescence based on signal strength of the fluorescence.

4. The fluorescence imaging device as recited in claim 3,
wherein the timing detection means is configured to detect that it has become a timing at which the signal strength has reached a maximum value based on the signal strength of the fluorescence.

5. The fluorescence imaging device as recited in claim 3,
wherein the timing detection means is configured to detect a timing at which the signal strength of the fluorescence becomes equal to or more than a threshold value based on the signal strength of the fluorescence.

6. The fluorescence imaging device as recited in claim 2,
wherein the timing detection means is configured to detect the timing for extracting the image of the fluorescence based on an operation input by a user.

7. The fluorescence imaging device as recited in claim 1,
wherein the extraction unit is configured to extract, centering on the timing for extracting the image of the fluorescence, the image of the fluorescence in the predetermined time range from a first time before the timing for extracting the image of the fluorescence to a second time after the timing for extracting the image of the fluorescence.

8. The fluorescence imaging device as recited in claim 1,
wherein the image acquisition unit is provided with a first light source unit configured to emit excitation light and a first detection unit configured to detect the fluorescence.

9. The fluorescence imaging device as recited in claim 1,
wherein the image acquisition unit is configured to acquire an image of visible light, and
wherein the fluorescence imaging device further comprises an image synthesizing unit configured to generate an image for reproduction in which the image of the fluorescence extracted by the extraction unit and the image of visible light reflected by the subject and extracted by the extraction unit are superimposed.

10. The fluorescence imaging device as recited in claim 9,
wherein the image acquisition unit is further provided with a second light source unit for emitting the visible light and a second detection unit for detecting the visible light reflected by the subject.

11. The fluorescence imaging device as recited in claim 1, further comprising:
a temporary storage unit configured to temporarily store an image acquired by the image acquisition unit at a time corresponding to the predetermined time range.

12. The fluorescence imaging device according to claim 1, further comprising:
a recording unit configured to record an image extracted by the extraction unit and an image generated from the image extracted by the extraction unit.

13. A fluorescence imaging system comprising:
the fluorescence imaging device as recited in claim 1;
a recording device configured to record an image for reproduction generated by the fluorescence imaging device; and
a display device configured to display the image for reproduction.
